(19) United States
(12) Patent Application Publication
(10) Pub. No.: US 2022/0044805 A1
(43) Pub. Date: Feb. 10, 2022

(54) Method for providing wearable device-collected physiological information to relevant service providers

(71) Applicant: Stephen Pickell, Lee's Summit, MO (US)

(72) Inventor: Stephen Pickell, Lee's Summit, MO (US)

(21) Appl. No.: 17/397,836

(22) Filed: Aug. 9, 2021

Related U.S. Application Data
(60) Provisional application No. 63/063,424, filed on Aug. 9, 2020.

(51) Int. Cl. G16H 40/67; G16H 40/63; G06F 1/16; H04L 29/06

(52) U.S. Cl. CPC G16H 40/67; H04L 63/123; G06F 1/163; G16H 40/63

(57) ABSTRACT

A method and system for capturing physiological information with wearable technology devices, transmitting that information to a data hub, and sharing that information with certain service providers who can use that data to determine availability and pricing of the services and who are authorized to receive the data collected from certain wearable device users.

This non-provisional application claims the benefit of provisional application No. 63/063,424 filed Aug. 9, 2020.

### BACKGROUND OF THE INVENTION

Wearable technology devices are relatively recent phenomenon that can be quite useful in, among other things, enabling individuals to monitor aspects of their personal health and physical fitness in real time. As wearable technology advances, the number and reliability of the multitude of health metrics that such technology can capture is ever increasing. Therefore, to the extent that wearable device users alter their behaviors in reaction to the physiological feedback they receive from the devices, wearable technology can positively impact health and wellness in ways that traditional physician consultations do not lend themselves to doing. That notion is primarily attributable to the fact that wearable devices can provide health information both immediately and continuously—enabling their users (and their physicians) to contextualize the information captured at any given moment, rather than having to extrapolate from and speculate based on isolated in time snapshots of health data captured from traditional medical examinations. It may also be partly due to some wearable devices being able to capture health data and detect impending physiological events of the type that are simply unlikely to be observed during or projected by a type of physician examination that is not intended to be focused on a specific area(s) of health.

Accordingly, wearable devices can contribute towards longer, healthier lives—an obvious benefit from the perspective of their end users. And from the perspective of insurers and other service providers whose business models involve assessing the health and wellness of those same end users, wearable devices can provide them with information of the types and breadth that can appreciably improve the quality of their business decision-making.

As with any type of technology, wearable devices adapted to capture health-related data are not uniform in their capabilities. Today, there are many brands, like FITBIT, APPLE, GARMIN, EMPATICA and WHOOP, under which several different models of wrist-worn and other wearable devices are produced that have widely varying capabilities. These modern wearable devices are designed to capture a wide and eclectic range of physiological data metrics, such as heart rate, sleep time, calories burned, steps walked, and skin temperature to name just a few examples. So, given variations in the types of physiological data that can be captured and reported by different contemporary wearable devices, different wearables tend to appeal to different end consumers based on their varied personal interests and health objectives.

By contrast, entities that are engaged in the business of health risk assessment, such as companies providing health, disability, and life insurance policies, might have a use for more and different health information than many of their consumers are consciously interested in discovering, and they certainly have incentive to analyze a wider breadth of information—wider with respect to both the types of and time points at which physiological data metrics are captured—than they, historically, have had access to.

Accordingly, the present inventor recognizes that much of the universe of physiological information that can be captured by modern wearable devices could be not only interesting to wearable end users, but also commercially beneficial to entities whose business it is to apply actuarial science and make data-driven determinations as to consumer eligibility and pricing of various insurance and other consumer products. Health insurance and life insurance providers, for example, are almost certainly better served by performing their requisite risk analyses using inherently unbiased data of the type captured by reliable wearable devices and transmitted to them electronically, than they are by performing those analyses using what may be less reliable and, likely, less comprehensive information related to them by persons—be those persons prospective insurance consumers, themselves, or even their well-trained physicians and therapists.

So, the present inventor recognizes a need for a process in which a trusted hub, or “clearinghouse,” entity receives physiological information about consumers that was collected by recognized wearable technology devices, authenticates the information, transforms the information in some fashion (in some instances), and then provides that raw or transformed information to service providers (with the consumers' consent) who are subscribers to the information sharing process and for whom such information may be useful in determining things like eligibility for and/or pricing of their products to those specific consumers. In so doing, the present inventor recognizes the need to ensure that the consumer health information it receives from wearable devices and shares with these service providers be authenticated as the physiological data of the specific consumer that it has associated with a specific wearable device (as opposed to information collected from another individual who may happen to wear that same device at a given moment).

Finally, the present inventor recognizes a further need for such a data clearinghouse service to provide and broker financial incentives to the stakeholders associated with that process (i.e., the wearable device-using consumer who is an existing or potential consumer of the subscribing service provider(s), the wearable device maker, and the subscribing service provider) in a manner that encourages stakeholder participation in the process and encourages that a greater breadth and sophistication of physiological data be collected and shared. The method of the present invention substantially fulfills these needs.

### SUMMARY OF THE INVENTION

The present invention generally relates to the sharing of wearable device-captured physiological data via at least one telecommunications network, and it is specifically directed to a subscription-based method for: (1) collecting such data from identified users of such devices that consent to its sharing; (2) authenticating that the collected data is that of the identified user; (3) transforming such collected and authenticated data in a way that it becomes more useful to certain types of service providers; and (4) sharing that transformed data with such service providers (e.g., insurance providers) that pay for access to the transformed data—presumably, to aid them in determining whether a consumer is eligible to receive their products and in determining identified consumer-specific pricing for such products.

In a preferred embodiment, the present method is a data clearinghouse that passes physiological information between wearable technology devices and certain service providers. It is anticipated that these services providers will be insurance providers (e.g., health insurers and life insurers), but they may be other businesses who deem consumer physiological information useful to their business models. It is also anticipated that consumers would indicate their consent (either directly to the clearinghouse, through a service provider application, or by way of entering into user agreements with makers of their wearable devices) to the clearinghouse's data collection and sharing activities and, possibly, to indicate which specific service providers the clearinghouse is permitted to share [specific items of] their physiological information with. Furthermore, it is anticipated that the clearinghouse's health information collection and sharing activities—and the corresponding consumer consents thereto—may vary from consumer to consumer and from service provider to service provider. For example, in one instance, the clearinghouse may be authorized to capture all of the health data metrics that are ever collected by a particular wearable device during a continuous timeframe (e.g., all metrics collected from Jan. 1, 2022 to Mar. 31, 2022). In that circumstance, it may logically follow that the service provider authorized to receive that collected information from the clearinghouse may pay the clearinghouse more (presumably, by way of recurring subscription fees) for that volume of information than it would pay for the same metrics captured only on the day of Jan. 1, 2022 (or for a continuous, but considerably shorter, one-week period).

In fact, it is in this regard that the present method promises to deliver to service providers more reliable, more relevant, and more dispositive consumer health and wellness information upon which they can base their service offerings and corresponding pricing to their consumers. By contrast, the health information that they have had to rely upon, heretofore, was limited, in both scope and volume, to only that which could be collected while a consumer was being observed by (and, typically, in the physical presence of) a physician or therapist. The present method, on the other hand, can produce and utilized information that is collected continuously over an indefinite timespan and is not subject to any subjective human characterization or manipulation, nor any selective non-disclosure that could distort what a body of collected health information might, otherwise, indicate to a service provider.

In a preferred embodiment of the present method, the clearinghouse would authenticate (or be able to verify that the wearing device, itself, authenticated) that the physiological data transmitted to it from a wearable device, in fact, was collected from the intended user that has previously authorized the clearinghouse to share his or her information. It is understood in the art of wearable device technology that such authentication can be accomplished by any of various biometric recognition mechanisms (e.g., analyzing bodily fluids, voice recognition, vein mapping, etc.)

In a preferred embodiment of the present method, the clearinghouse would transform the data received from consumers in some manner. Now, such a “transformation” could concern merely the ways in which collected data metrics are compiled and formatted for sharing, or it could involve algorithmically producing various outputs using certain received data metrics as inputs. In any case, the transformed data is to be provided to service providers that pay a [recurring] fee for access to such transformed information.

Finally, in a preferred embodiment, the wearable device users, the wearable device makers, and the service providers are all incentivized to participate in the process. For example, the user may be incentivized in the form of a lower priced offer for an insurance product than he would otherwise receive if he did not participate in the process. The device maker may be incentivized to capture and transmit more or certain types of physiological metrics in the form of payment from the clearinghouse (presumably, a share of the subscription fees collected by the clearinghouse from service providers). The service providers, as previously mentioned, are incentivized to participate by way of recognizing that they can make more prudent financial decisions on consumer eligibility and pricing using the higher quality transformed data that originates from wearable technology, rather than relying on statements from consumers or even their attending physicians.

### BRIEF DESCRIPTION OF THE DRAWINGS

FIG. 1 is a schematic diagram illustrating a system for sharing wearable device-collected physiological information to service providers that the wearable device users may happen to contract with.

### DETAILED DESCRIPTION OF THE PREFERRED EMBODIMENTS

This disclosure, as defined by the claims that follow and as illustrated, by way of example, in the accompanying drawing, relates to a method of disseminating physiological data captured by wearable technology (including useful data that may be derived from desired manipulation of such captured data), through a telecommunications network, to healthcare insurers, life insurers, and other service providers that utilize such consumer health data in making risk assessments to determine specific customer eligibility and pricing for their insurance products and other services. The present inventor anticipates that a central clearinghouse will facilitate this method and that the aforementioned types of service providers will participate in it for the reason that it provides them information in a manner that can supplement, if not supplant, the traditional practice of obtaining [prospective] customer health information by way of obtaining records of those customers' medical examinations—health information which, typically, reflects mere momentary snapshots of observation and is less comprehensive than that which can be collected via the present method.

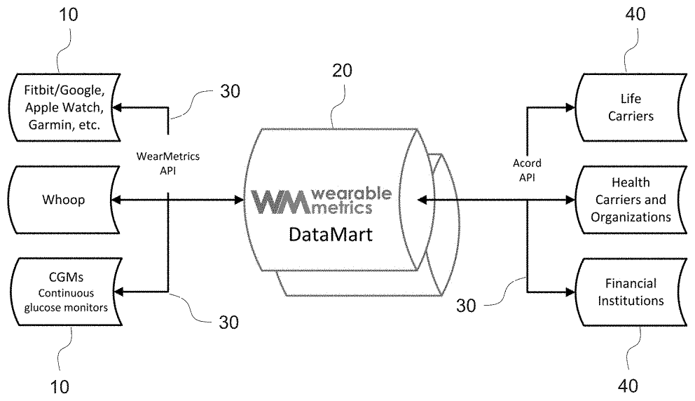

As indicated in FIG. 1, the present method for sharing wearable device-captured data concerning health and wellness within a system comprises at least three elements:

(1) wearable technology devices 10 to be worn by [prospective] consumers whose health and fitness is of interest and who explicitly consent to the health and wellness information captured by their wearable devices being shared with entities with which they may contract to obtain insurance products and other risk assessment-based services (hereinafter, broadly referred to as simply “services”);
(2) a clearinghouse 20 that operates a data hub which, through at least one telecommunications network 30, (i) receives consumer physiological data transmitted by wearable devices 10; (ii) authenticates that that data was collected from a person who has consented to that data being disclosed to a third party provider of the services; (iii) in some instances, transforms the received data in ways that make it more useful to a service provider; and (iv) transmits that raw and/or transformed data to a service provider(s); and
(3) service providers 40 who sell the services and who determine the customer-specific viability and pricing of the services, at least in part, in accordance with risk assessments made using physiological information that they receive from the clearinghouse 20.

Many types of wearable devices 10 can be utilized within the scope of the present method. What is pertinent is that a given wearable device 10 be capable of capturing information, about its user, that is useful to a service provider 40 in its task of analyzing the risks associated with offering its product to that device user and, thereby, determining an appropriate product offering price. This information can include heart rate, blood pressure, and various types of health monitoring, in addition to various types of activity tracking. Furthermore, each wearable technology-using consumer of service provider products (e.g., health insurance policy owner, life insurance policy owner, etc.) can utilize more than one wearable device in order that a broader range of his physiological information may be captured and disclosed (to the extent that no one model of wearable device 10 is equipped to capture all the various types of information that is, in fact, capturable by modern wearable technology and is of interest to the service providers 40 within the present system).

In the context of the present disclosure, the “clearinghouse” 20 jointly refers to a server (hereinafter, itself, also referred to simply as a host “computer”) and a web interface (i.e., website and/or web application) stored thereon. The telecommunications network 30 through which the computer and web interface 20 are accessed by consumers' wearable devices 10 and other terminals (e.g., personal computers, smart devices, etc.) will typically be the Internet, but it may be any network through which the host computer 20 can receive data from wearable devices 10 and send that data to remote terminals—including those of the service providers 40.

In addition to simply passing along information received from wearable devices 10, in a preferred embodiment, the clearinghouse computer 20 may contain software for converting data received from wearable devices 10 into derivative data that is even more pertinent to a particular service provider's risk analysis. For example, the clearinghouse 20 may perform a comparative analysis of the same type of health data that was collected, by a wearable device 10, at two separate timepoints and report any data deviations to a service provider 40. Or the clearinghouse 20 might be programmed to derive particular conclusions from comparing multiple types of physiological data and, then, transmit those derived conclusions to a service provider(s) 40 via the network 30.

Finally, the service providers 40 are healthcare providers, life insurers, and any other entities that can benefit from receiving the physiological information from wearable devices 10. However, it is anticipated that only those specific service providing entities that a wearable device user has consented to receiving its physiological information will, in fact, receive that user's information via the clearinghouse 20.

An iteration of the preferred method of the present method is executed by, first, a wearable device user indicating consent to one or more service providers 40 having access to some or all of the physiological information that his model of wearable device 10 is capable of capturing from him—where the distinction between what physiological information is authorized to be shared and what is not might be based on the specific type of physiological information in question or when the physiological information is collected. For example, a user might consent to sharing heart rate data that is to be captured in October, but not blood pressure data that is to be captured in November. In any event, typically, this would be done by the user completing a consent form that is presented, in a web interface and via the network 30, to his terminal by the clearinghouse 20.

In conjunction with submission of these consents, certain biometric information collected by the user's wearable device 10 may be submitted to and stored at the clearinghouse 20 for comparison with comparable biometric information to be received from that same wearable device 10 in the future—the purpose being to authenticate that physiological data later received from that particular wearable device 10 is from the same consenting user previously associated with that device 10 (as opposed to another individual who may happen to wear that device 10 in the future). Later received unauthenticated data can be discarded by the clearinghouse 20, while authenticated data is shared with the service provider(s) 40 (if consented to).

Then, going forward, as physiological information is captured by the wearable device 10, it is transmitted to the clearinghouse 20, authenticated as being from user previously associated with that device 10, and transmitted (in its collected or a transformed form) to service providers 40 that are authorized to receive the user's information.

It is anticipated that the present method will operate on a business model in which several economic factors come to bear. For example, consumer-users are incentivized, or maybe even required, to both use wearable devices 10 capable of capturing certain of their health and wellness information (the “physiological information”) and authorize that such captured physiological information be transmitted to the clearinghouse 20 to be shared with service providers 40 (either in raw collected form or some modified derivative form). To wit, a health insurance service provider 40 may require that it receive certain physiological data points during a certain timeframe and with a certain data collection interval frequency as a precondition to even offering a health insurance policy to a consumer.

So, when a wearable device user does provide access to at least a certain amount of his physiological information via the present system, the clearinghouse 10 may, through its web interface, indicate to that user that he is, consequently, eligible to purchase a certain service from a service provider(s) and that the cost of such service is lower to him than it otherwise might have been.

For another example, it is anticipated that the service providers 40 will compensate the clearinghouse 10 for facilitating their receipt of this consumer health and wellness information, as access to the types and volume of such information that can be captured by modern wearable technology can vastly improve the service providers' risk assessment capabilities—thereby leading them to make better financial decisions concerning consumer eligibility and consumer-specific pricing of their services. It is further anticipated that this compensation to the clearinghouse 20 will be in the form of recurring subscription fees paid to it by the service providers 40, and that these subscription fee amounts will be predicated on factors like the number of discrete consumers for which information is provided, the types of physiological information that is provided, and the volume of such information that is provided (in terms of how many discrete types of information from each consumer are captured by their wearable devices 10 and passed along to the service providers 40)

Finally, for another example, it is conceivable that the manufacturers of wearable device technology might receive an appreciable benefit—in the form of cross-promotion of their wearable technology or direct monetary payment to them—from the service providers 40. Likely, such benefit would be predicated on the type and or volume of physiological information that their manufactured devices are able to capture and share via the system.

It is understood that substitutions and equivalents for and combinations of various elements set forth above may be obvious to those skilled in the art and may not represent a departure from the spirit of the invention. Therefore, the full scope and definition of the present invention is to be set forth by the claims that follow.

What is claimed is:

1. A method for providing wearable device users' physiological information, to a service provider, using a computing device and telecommunications network(s), the method comprising:
receiving physiological information from a wearable device via a telecommunications network;
authenticating that that received physiological information was collected from a wearable device user that has indicated consent to share its physiological information with the service provider;
providing the authenticated physiological information to the service provider via a telecommunications network.

2. The method of claim 1, wherein authenticating comprises comparing (a) previously collected biometric information associated with said user with (b) biometric information later collected by said wearable device.

3. The method of claim 1, wherein said user consents to share, with said service provider, all physiological information collected by said wearable device.

4. The method of claim 1, wherein said user consents to share, with said service provider, only physiological information that is collected, by said wearable device, during a predetermined time period(s).

5. The method of claim 1, further comprising receiving compensation, from said service provider, for sharing physiological information therewith.

6. The method of claim 1, further comprising conferring a benefit, to the maker of said wearable device, in response to receiving a certain quantity or type of authenticated physiological information from said wearable device.

7. The method of claim 1, further comprising indicating a benefit, to said user, in response to receiving a certain quantity or type of authenticated physiological information from said wearable device.

8. The method of claim 7, wherein said indicated benefit is defined by at least one of: (a) a discounted cost of a service provided by said service provider; and (b) eligibility for receiving a product provided by said service provider.

9. A method for providing wearable device-collected physiological information, to a service provider, using a computing device and a telecommunications network(s), the method comprising:
receiving compensation, from said service provider, for sharing physiological information therewith;
receiving, from a wearable device via a telecommunications network, physiological information collected from a wearable device user which has indicated consent to share its physiological information with the service provider;
generating, at the computing device, a transformation of the received physiological information, wherein the transformed information comprises or derives from the received physiological information;
providing the transformed information to the service provider via a telecommunications network.

10. The method of claim 9, further comprising conferring a benefit, to the maker of said wearable device, in response to receiving a certain quantity or type of physiological information from said wearable device.

11. The method of claim 9, further comprising indicating a benefit to said user in response to receiving a certain quantity or type of physiological information from said wearable device.

12. The method of claim 11, wherein said indicated benefit is defined by at least one of: (a) a discounted cost of a service provided by said service provider; and (b) eligibility for receiving a product provided by said service provider.
